(19) United States
(12) Patent Application Publication
(10) Pub. No.: US 2015/0177232 A1
(43) Pub. Date: Jun. 25, 2015

(54) Clinical Specimen Detector

(71) Applicant: National Tsing Hua University, Hsin Chu City (TW)

(72) Inventors: Hsi-Kai Wang, NTaipei City (TW); Shu-Ting Fan, New Taipei City (TW); Hong-Ren Lin, New Taipei City (TW); Ting-Chang Chang, Taoyuan County (TW); Chia-Chi Wu, Yilan City (TW); Chao-Min Cheng, New Taipei City (TW)

(73) Assignee: National Tsing Hua University, Hsin Chu City (TW)

(21) Appl. No.: 14/256,988

(22) Filed: Apr. 20, 2014

(30) Foreign Application Priority Data
Dec. 20, 2013 (TW) 102147545

(51) Int. Cl. G01N 33/53

(52) U.S. Cl. CPC G01N 33/5302

(57) ABSTRACT

A clinical specimen detector includes a clip having a clamping end and a pressing end; and wherein a pair of compressing slices are disposed on the clamping end, and a channel is disposed in the lower end of at least one of the pair of compressing slices. A detecting reagent test chip is attached to the outer surface of the compressing slice. The lower end of the detecting reagent test chip is substantially aligned with and is adjacent to the channel. The lower ends of the compressing slices clamp an adsorptive material adsorbed with body fluid such that the body fluid can be released from the adsorptive material because of the compression of the compressing slices and can be delivered to the lower end of the detecting reagent test chip via the channel, so as to perform disease testing and physiological condition testing with the detecting reagent test chip.

### CROSS-REFERENCE TO RELATED APPLICATIONS

This present application claims priority to TAIWAN Patent Application Serial Number 102147545, filed on Dec. 20, 2012, which is herein incorporated by reference.

### TECHNICAL FIELD

The present invention relates to clinical detection, and more particularly to a clinical specimen detector.

### BACKGROUND OF RELATED ART

Recently, the common clinical specimen sampler, for example the endometrial cancer specimen sampler, generally includes suction tubes for uterus cavity, uterus cavity brushes, plastic spatulas for uterus cavity, uterus cavity currettes, etc. The way to utilize them is to place them into the uterus cavity, and suck or currette the surface of the endomembrane to obtain the endomembrane cell and its secretion, so as to further process and test the endomembrane cell and its secretion to find cancel cells. The positive rate thereof may reach more than 90%. This method can be executed at the clinic, and no operation or anesthesia is needed, but the method can be utilized only for screening. A final definite diagnosis still must be made according to pathological examination results.

However, the endomembrane cell and its secretion obtained by the traditional endometrial cancer specimen sampler mentioned above often mix with each other. Therefore, if it is desired to test the secretion, the endomembrane cell and the secretion need to be separated first. Thus, the pretreatment procedure before directly testing the secretion will be complicated, and it will be difficult to test the secretion directly. Besides, the traditional endometrial cancer specimen sampler mentioned above often hurts the test subject or makes the test subject feel quite uncomfortable when sucking or curretting the surface of the endomembrane to obtain the endomembrane cell and its secretion, which renders the test subject to refuse the usage of the traditional endometrial cancer specimen sampler.

Therefore, there is still a need for a technical solution which can solve the shortcomings of the traditional clinical specimen sampler and the difficulty of directly testing the secretion.

### SUMMARY

To overcome the shortcomings of the traditional clinical specimen sampler and the difficulty of directly testing the secretion, the present invention provides a novel clinical specimen detector.

In one aspect, the present invention discloses a clinical specimen detector, including a clip having a clamping end and a pressing end; and wherein a pair of compressing slices are disposed on the clamping end, and a channel is disposed in the lower end of at least one of the pair of compressing slices. A detecting reagent test chip is attached to the outer surface of at least one of the pair of compressing slices. The lower end of the detecting reagent test chip is substantially aligned with the channel and is adjacent to the channel. The lower ends of the compressing slices may clamp an adsorptive material adsorbed with body fluid such that the body fluid can be released from the adsorptive material because of the compression of the pair of compressing slices and can be delivered to the lower end of the detecting reagent test chip via the channel, so as to perform disease testing and physiological condition testing by utilizing the detecting reagent test chip.

In one embodiment, the clip may include a first clamping element and a second clamping element, and the first clamping element is pivot jointed with the second clamping element at the center of the opposing side surfaces of the first clamping element and the second clamping element. In one embodiment, free ends of the first clamping element and the second clamping element form the pressing end. In one embodiment, the other ends of the first clamping element and the second clamping element form the clamping end.

In one embodiment, the clip may further include an elastomer, and the clamping ends of the first clamping element and the second clamping element are resiliently leaned against each other through the elastomer. In one embodiment, the elastomer may include a C shaped elastomer. In another embodiment, the elastomer may include a flat-arch shaped elastomer.

In one embodiment, an adsorbable material which can release the sampled specimen slowly may be disposed on the inner wall of the channel to assist in delivering the body fluid from the adsorptive material to the lower end of the detecting reagent test chip. In one embodiment, the detecting reagent test chip may include but be not limited to lateral-flow immunoassay test strip or bio-chip.

One advantage of the present invention is that the clinical specimen detector of the present invention can omit the step of obtaining the body fluid from the sampling adsorber or the adsorptive material and can directly show the test result about whether the test subject has a specific disease or the physiological condition of the test subject.

Another advantage of the present invention is that the detecting speed of the clinical specimen detector of the present invention is fast, it is easy to operate the clinical specimen detector of the present invention and the cost of the clinical specimen detector of the present invention is cheap.

These and other advantages will become apparent from the following description of preferred embodiments taken together with the accompanying drawings and the appended claims.

### BRIEF DESCRIPTION OF THE DRAWINGS

The present invention may be understood by some preferred embodiments and detailed descriptions in the specification and the attached drawings below.

FIG. 1 illustrates a diagram of a clinical specimen sampler of the present invention in accordance with one embodiment of the present invention;

FIG. 2 illustrates a flow chart showing a method for utilizing the clinical specimen sampler of the present invention in accordance with one embodiment of the present invention;

FIG. 3 illustrates a front view of the clinical specimen detector of the present invention in accordance with one embodiment of the present invention;

FIG. 4 illustrates a side view of the clinical specimen detector of the present invention in accordance with one embodiment of the present invention;

FIG. 5 illustrates a diagram of the clinical specimen detector of the present invention in accordance with one embodiment of the present invention;

FIG. 6 illustrates a flow chart showing a method for employing the clinical specimen detector of the present invention in accordance with one embodiment of the present invention; and

FIG. 7 illustrates a flow chart of partial views showing a method for employing the clinical specimen detector of the present invention in accordance with one embodiment of the present invention.

### DETAILED DESCRIPTION

The present invention will now be described with the preferred embodiments and aspects and these descriptions interpret structure and procedures of the present invention only for illustrating but not for limiting the Claims of the present invention. Therefore, except the preferred embodiments in the specification, the present invention may also be widely used in other embodiments.

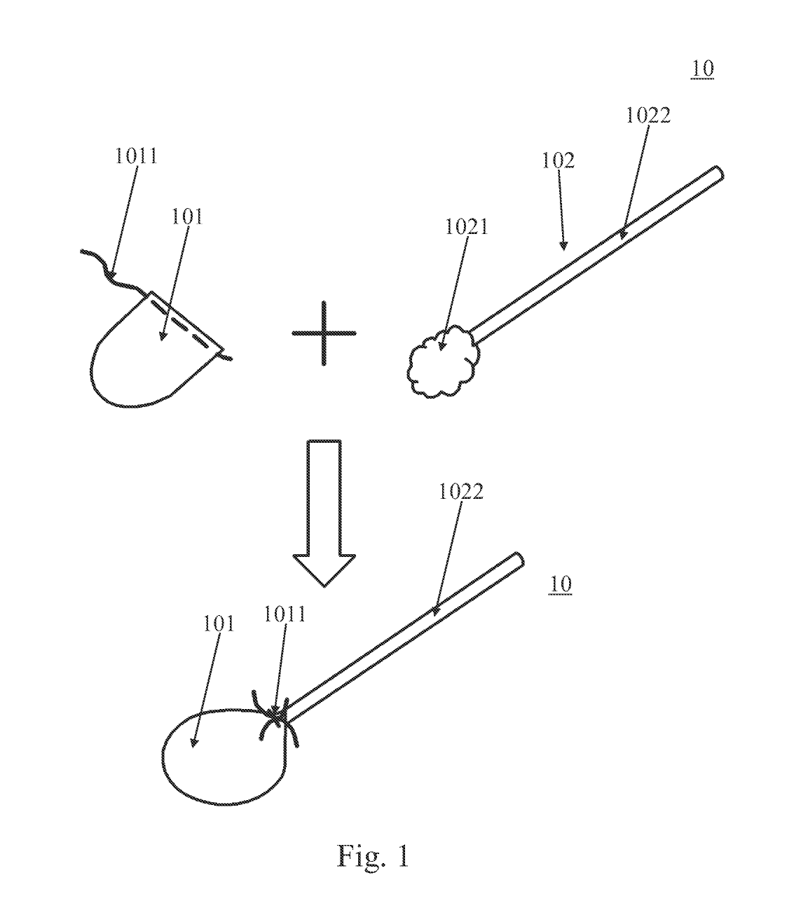

The present invention provides a clinical specimen sampler and a clinical specimen detector. The clinical specimen sampler of the present invention may be applied to any diseases which can be tested via body fluids, for example but be not limited to, the endometrial cancer, or may be utilized to detect the physiological condition of the test subject, for instance but be not limited to, the ovulation cycle of woman. In one embodiment of the present invention, as shown in FIG. 1, the clinical specimen sampler 10 of the present invention may include a filtering device 101 and a sampling device 102. In one embodiment, the filtering device 101 may be a filter screen. In one specific embodiment, the material of the filtering device 101 may include but be not limited to gauze, non-woven fabric paper towel, non-woven fabric bandage or the equivalent thereof, so as to filter out the substance except the body fluid, such as the mucosal tissue, prevent the mucosal tissue from entering the sampling adsorber of the sampling device, and enable the body fluid secreted by an intima of an organ to enter the sampling adsorber of the sampling device, so as to be absorbed by the sampling adsorber. In a preferred embodiment, the material of the filtering device 101 may preferably be the non-woven fabric paper towel because it is much comfortable to the test subject. The size of the mesh of the filtering device may be at least smaller than the size of the mucosal tissue, so as to filter out the mucosal tissue. In one embodiment, the filtering device 101 may be bag-shaped. In one embodiment, a binding wire 1011 may be disposed around the bag opening of the filtering device 101 to fix the filtering device 101 onto the sampling adsorber (which will be described in detail hereinafter) of the sampling device 102. In one embodiment, as shown in FIG. 1, the sampling device 102 may include a sampling adsorber 1021 and a rod 1022. The sampling adsorber 1021 is connected with one end of the rod 1022. In one embodiment, the material of the sampling adsorber 1021 may include but be not limited to sponge block, cotton ball or the equivalent thereof. In one preferred embodiment, the material of the sampling adsorber 1021 may preferably be cotton ball. The sampling adsorber 1021 can adsorb the body fluid secreted by the intima of an organ, for instance but be not limited to, the endometrium, the inner membrane of the oral cavity, so as to perform disease testing or physiological condition testing while the rod 1022 can be held by the user. In one embodiment, the material of the rod 1022 may, for instance, include but be not limited to wooden rod, bamboo rod, plastic rod or the equivalent thereof. In one embodiment, a special physically separable design, for example but be not limited to a breaking notch, may be formed on the rod 1022, such that the user can apply force to the breaking notch to break the rod easily and separate the sampling adsorber 1021 and the rod 1022 easily, so as to further process the sampling adsorber 1021. The size of the filtering device 101 is slightly larger than the size of the sampling adsorber 1021 of the sampling device 102, such that the sampling adsorber 1021 can be encompassed in the filtering device 101 and the binding wire 1011 can be employed to tightly fix or bind the filtering device 101 to form the clinical specimen sampler 10 as shown in FIG. 1. In one embodiment, a lubricant, for instance but be not limited to, vaseline may be coated on the outer surface of the filtering device 101 encompassing the sampling adsorber 1021 to enhance the comfortability to the test subject when applying the clinical specimen sampler 10 to the test subject.

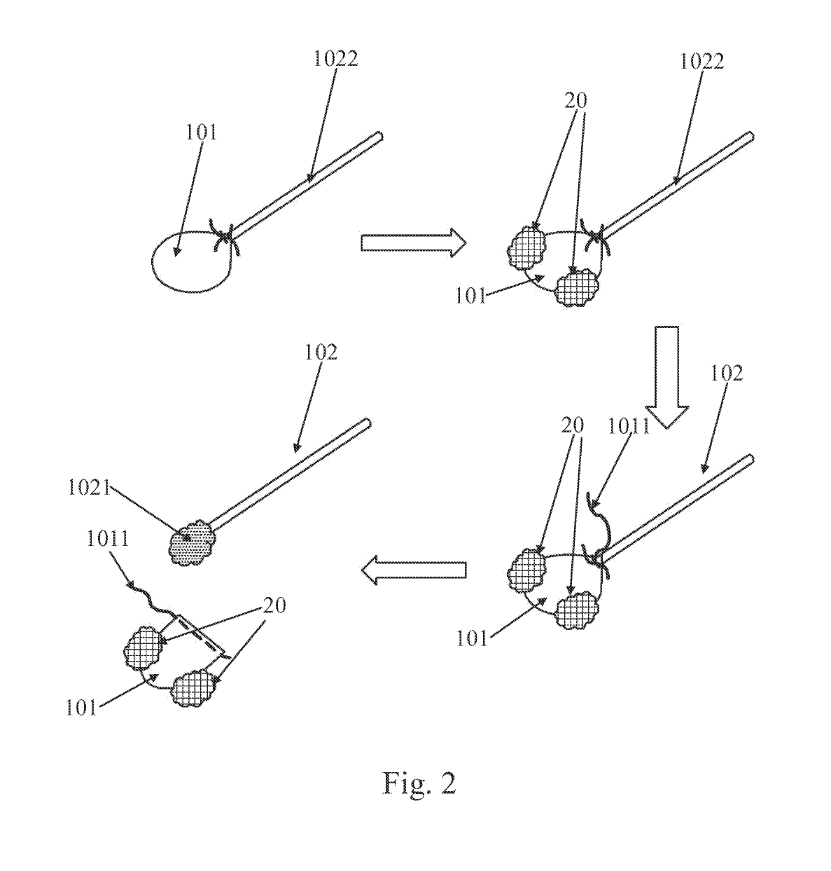

Please refer to FIG. 2, which illustrates a method for utilizing the clinical specimen sampler of the present invention. First, the user may hold the rod 1022 and render the sampling adsorber encompassed in the filtering device 101 to contact the surface of the intima of the organ, for instance the endometrium, the inner membrane of the oral cavity, etc of the test subject, to adsorb the body fluid secreted by the intima of the organ. While the sampling adsorber adsorbs the body fluid secreted by the intima of the organ, some substances except the body fluid, for example the mucosal tissue 20, may usually be adhered to the filtering device 101. These mucosal tissues 20 might be unnecessary for the disease testing or physiological condition testing and cannot pass the filtering device 101 to enter the sampling adsorber because the size of these mucosal tissues 20 is larger than the size of the mesh of the filtering device 101. Subsequently, as shown in FIG. 2, the user may loosen the binding wire 1011 and separate the sampling device 102 and the filtering device 101, such that the sampling device 102 adsorbed with the body fluid can be separated from the filtering device 101 adhered with the mucosal tissue 20. The sampling adsorber 1021 of the sampling device 102 adsorbed with the body fluid may be employed to perform the disease testing and/or the physiological condition testing by utilizing the clinical specimen detector of the present invention while the filtering device 101 adhered with the mucosal tissue 20 may be disposed of or be utilized to perform other analyses. Therefore, the clinical specimen sampler of the present invention can sample the body fluid secreted by the intima of the organ, and the sampled body fluid will not be mixed with the substances other than the body fluid, such as the mucosal tissue, such that no additional steps are needed to remove the mucosal tissue when performing testing.

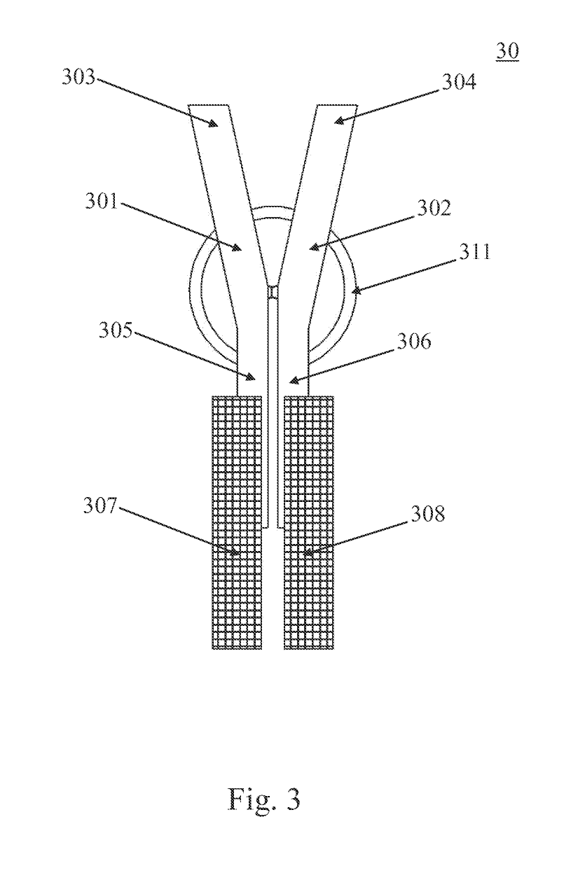

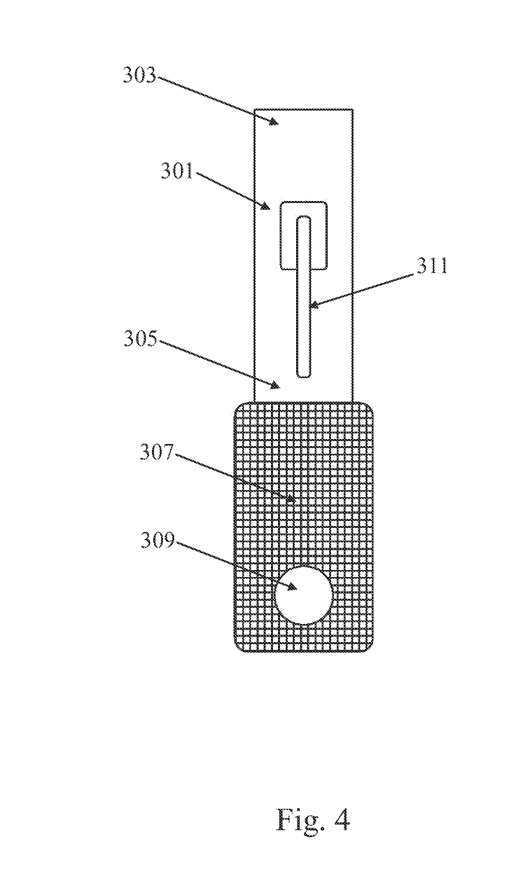

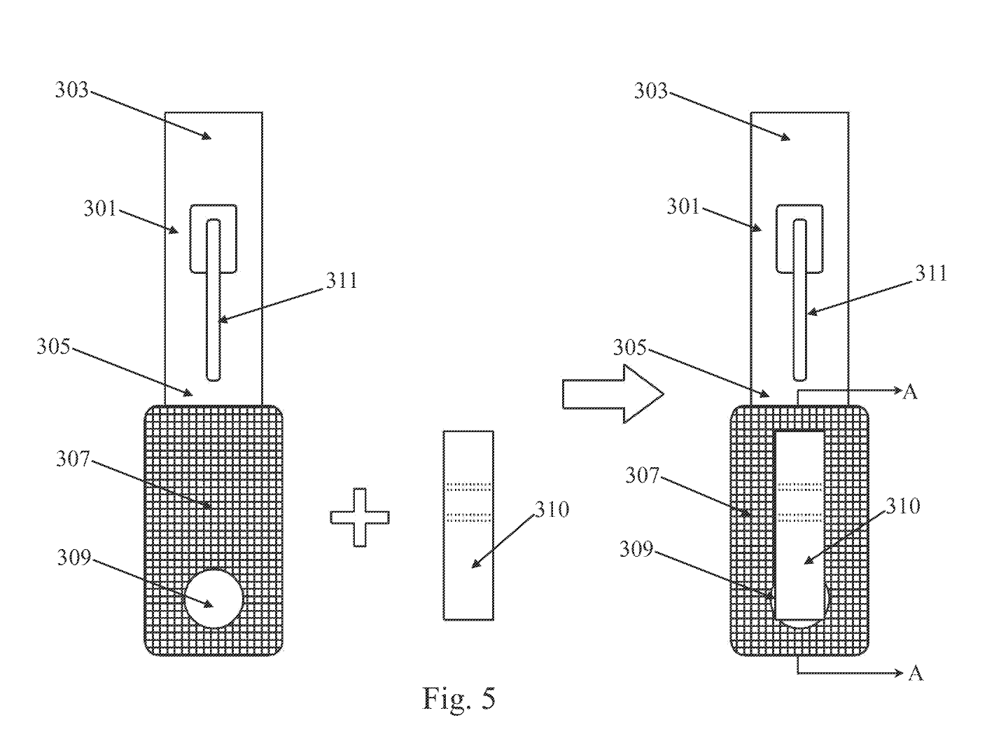

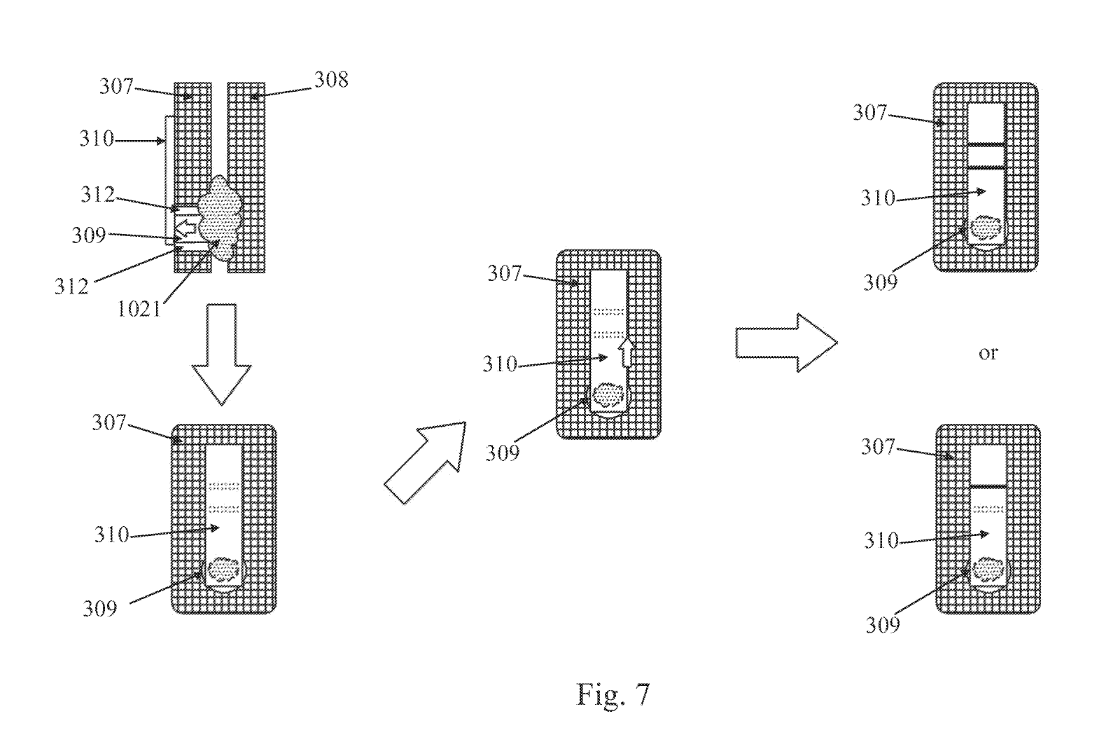

In one embodiment, the sampling adsorber 1021 of the sampling device 102 adsorbed with the body fluid may be employed to perform the disease testing and/or the physiological condition testing to test whether the test subject has a specific disease, for instance endometrial cancer, etc or to test the physiological condition, for example but be not limited to the ovulation cycle of woman, of the test subject by utilizing the clinical specimen detector of the present invention. FIG. 3 illustrates a front view of the clinical specimen detector of the present invention in accordance with one embodiment of the present invention. FIG. 4 illustrates a side view of the clinical specimen detector of the present invention in accordance with one embodiment of the present invention. As shown in FIG. 3, in one embodiment, the clinical specimen detector 30 of the present invention may include a first clamping element 301 and a second clamping element 302, and the first clamping element 301 is pivot jointed with the second clamping element 302 at the center of the opposing side surfaces of the first clamping element 301 and the second clamping element 302. Free ends of the first clamping element 301 and the second clamping element 302 form pressing ends 303, 304 while the other ends of the first clamping element 301 and the second clamping element 302 form clamping ends 305, 306. The clamping ends 305, 306 of the first clamping element 301 and the second clamping element 302 are resiliently leaned against each other through an elastomer 311 (for example but be not limited to C shaped elastomer, flat-arch shaped elastomer), and a pressing and clamping action can be performed by pressing and releasing the pressing ends 303, 304 of the first clamping element 301 and the second clamping element 302. A compressing slice 307, 308 is respectively disposed on the clamping ends 305, 306. As shown in FIG. 4, a channel 309 is disposed in the lower end of the compressing slice 307 and is formed through the compressing slice 307. As shown in FIG. 5, a detecting reagent test chip 310, for instance but be not limited to lateral-flow immunoassay test strip or bio-chip, may be attached to the outer surface of the compressing slice 307. The lower end of the detecting reagent test chip 310 may be substantially aligned with the channel 309 and may be adjacent to the channel 309. In one embodiment, as shown in the upper left portion of FIG. 7, an adsorbable material 312 which can release the body fluid slowly, for instance but be not limited to cotton, may be disposed on the inner wall of the channel 309 to assist in delivering the body fluid from the sampling adsorber or other adsorptive materials to the lower end of the detecting reagent test chip 310.

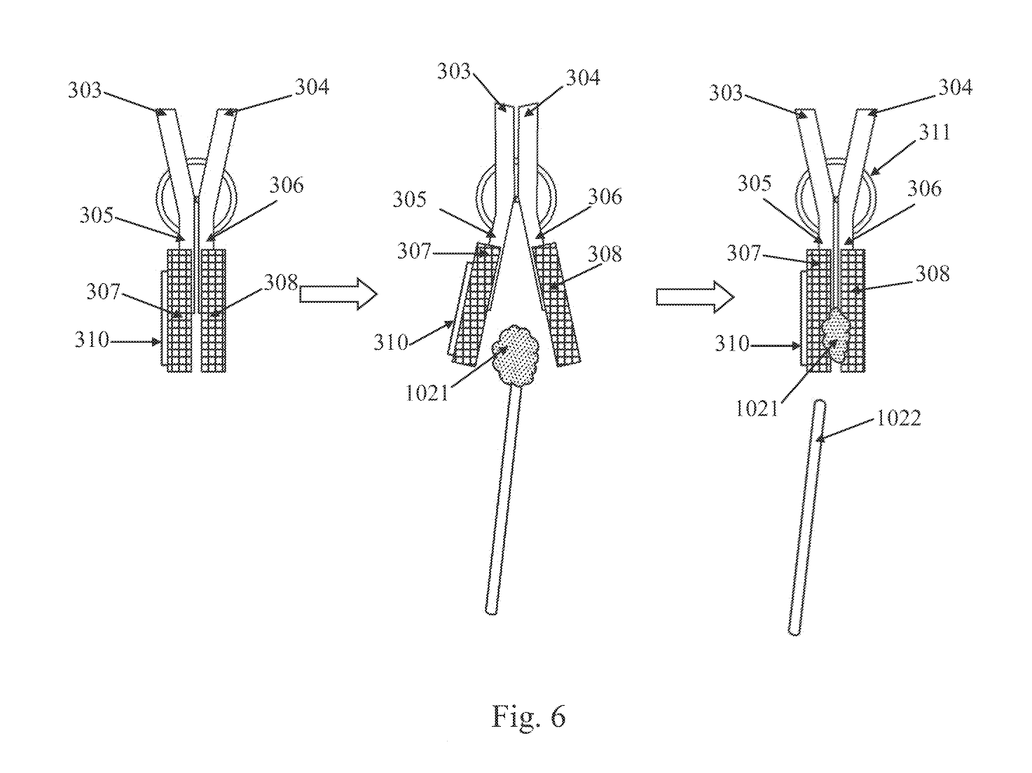

Please refer to FIGS. 6 and 7, which illustrate a method for employing the clinical specimen detector of the present invention. First, the pressing ends 303, 304 of the clinical specimen detector are pressed to increase the distance between the clamping end 305 and the clamping end 306 and the distance between the compressing slice 307 and the compressing slice 308, i.e. open the clamping ends 305, 306 and the compressing slices 307, 308 thereon. Subsequently, the sampling adsorber 1021 of the sampling device 102 adsorbed with the body fluid is placed between the lower ends of the compressing slices 307, 308. Then, the pressing ends 303, 304 are released to render the clamping ends 305, 306 and the compressing slices 307, 308 thereon back to the clamping position through the resilient force of the elastomer 311 (for example but be not limited to C shaped elastomer, flat-arch shaped elastomer, etc) and clamp the sampling adsorber 1021 between the lower ends of the compressing slices 307, 308. Subsequently, the sampling adsorber 1021 and the rod 1022 of the sampling device 102 are separated. FIG. 7 illustrates a partial view of the compressing slices 307, 308 and the detecting reagent test chip 310 of the clinical specimen detector in accordance with one embodiment of the present invention. The upper left portion of FIG. 7 is a partial sectional view taken along line A-A in the right portion of FIG. 5. The sampling adsorber 1021 is clamped between the lower ends of the compressing slices 307, 308. As shown in FIG. 7, after the sampling adsorber 1021 of the sampling device adsorbed with the body fluid is compressed by the compressing slices 307, 308, the adsorbed body fluid will be released from the sampling adsorber 1021, be adsorbed by the adsorbable material 312 which can release the body fluid slowly on the inner wall of the channel 309 and be delivered to the lower end of the detecting reagent test chip 310, for example but be not limited to lateral-flow immunoassay test strip or bio-chip. Subsequently, the body fluid moves from the lower end of the detecting reagent test chip 310, for example but be not limited to lateral-flow immunoassay test strip or bio-chip, toward the test zone of the detecting reagent test chip 310, for example but be not limited to lateral-flow immunoassay test strip or bio-chip, via capillarity, so as to show positive result (as shown in the upper right portion of FIG. 7) or negative result (as shown in the lower right portion of FIG. 7) to test whether the test subject has a specific disease or to test the physiological condition of the test subject. Therefore, the clinical specimen detector 30 of the present invention can omit the step of obtaining the body fluid from the sampling adsorber and can directly show the test result about whether the test subject has a specific disease or the physiological condition of the test subject. For instance, the step of rendering the body fluid in the sampling adsorber to enter the buffer solution or the steps of adding some buffer solution to the sampling adsorber adsorbed with the body fluid and obtaining the body fluid via centrifugation procedure can be omitted, such that the detecting procedure can be further simplified. Besides employing the sampling adsorber 1021 of the sampling device 102 adsorbed with the body fluid to perform the detecting procedure as disclosed above, the clinical specimen detector of the present invention can clamp any adsorptive materials adsorbed with the body fluid and the adsorptive materials are not limited to the sampling adsorber disclosed herein.

The foregoing description is a preferred embodiment of the present invention. It should be appreciated that this embodiment is described for purposes of illustration only, not for limiting, and that numerous alterations and modifications may be practiced by those skilled in the art without departing from the spirit and scope of the present invention. It is intended that all such modifications and alterations are included insofar as they come within the scope of the present invention as claimed or the equivalents thereof.

What is claimed is:

1. A clinical specimen detector, comprising:
a clip having a clamping end and a pressing end; and
wherein a pair of compressing slices are disposed on said clamping end and a channel is disposed in a lower end of at least one of said pair of compressing slices, a detecting reagent test chip being attached to an outer surface of said at least one of said pair of compressing slices, a lower end of said detecting reagent test chip being substantially aligned with said channel and being adjacent to said channel, wherein lower ends of said pair of compressing slices clamp an adsorptive material adsorbed with body fluid such that said body fluid is released from said adsorptive material because of compression of said pair of compressing slices and is delivered to said lower end of said detecting reagent test chip via said channel, so as to perform disease testing and physiological condition testing by utilizing said detecting reagent test chip.

2. The detector of claim 1, wherein said clip comprises a first clamping element and a second clamping element.

3. The detector of claim 2, wherein said first clamping element is pivot jointed with said second clamping element at a center of opposing side surfaces of said first clamping element and said second clamping element.

4. The detector of claim 2, wherein free ends of said first clamping element and said second clamping element form said pressing end.

5. The detector of claim 4, wherein the other ends of said first clamping element and said second clamping element form said clamping end.

6. The detector of claim 5, wherein said clip further comprises an elastomer, said clamping ends of said first clamping element and said second clamping element being resiliently leaned against each other through said elastomer.

7. The detector of claim 6, wherein said elastomer comprises a C shaped elastomer.

8. The detector of claim 6, wherein said elastomer comprises a flat-arch shaped elastomer.

9. The detector of claim 1, wherein an adsorbable material which can release said body fluid slowly is disposed on an inner wall of said channel to assist in delivering said body fluid from said adsorptive material to said lower end of said detecting reagent test chip.

10. The detector of claim 9, wherein said adsorbable material which can release said body fluid slowly comprises cotton.

11. The detector of claim 1, wherein said detecting reagent test chip comprises lateral-flow immunoassay test strip.

12. The detector of claim 1, wherein said detecting reagent test chip comprises bio-chip.
